(19) United States
(12) Patent Application Publication
(10) Pub. No.: US 2024/0412878 A1
(43) Pub. Date: Dec. 12, 2024

(54) PERSONALIZED INSIGHTS INTO TUMOR EVOLUTION AND AI-BASED TREATMENT DECISION SUPPORT SYSTEMS AND METHODS

(71) Applicants: FRED HUTCHINSON CANCER CENTER, Seattle, WA (US); UNIVERSITY OF WASHINGTON, Seattle, WA (US)

(72) Inventors: Elizabeth Krakow, Seattle, WA (US); Ivana Bozic, Seattle, WA (US); Nathan Lee, Germantown, OH (US); Cecilia Yeung, Mercer Island, WA (US); Jerald Radich, Sammamish, WA (US); Olga Sala-Torra, Seattle, WA (US); Isaac Jenkins, Seattle, WA (US)

(73) Assignees: FRED HUTCHINSON CANCER CENTER, Seattle, WA (US); UNIVERSITY OF WASHINGTON, Seattle, WA (US)

(21) Appl. No.: 18/738,564

(22) Filed: Jun. 10, 2024

Related U.S. Application Data
(60) Provisional application No. 63/507,047, filed on Jun. 8, 2023.

(51) Int. Cl. G16H 50/70; G06F 9/451; G16H 10/60

(52) U.S. Cl. CPC G16H 50/70; G06F 9/451; G16H 10/60

(57) ABSTRACT

A system generates a visualization that represents progression of a cancer genome. To generate the visualization, the system obtains a genetic dataset derived from cancer cell samples of a patient and clinical data associated with the patient. A genomic data structure is generated to represent the genetic dataset, and clinical event data structures are generated to represent each of a plurality of clinical events based on the clinical data. The visualization includes interactive elements that are populated based on the genomic data structure and the clinical event data structures. When a user input associated with a first interactive element is detected, the system displays information associated with the first interactive element based on the genomic data structure or the clinical event data structures.

### CROSS-REFERENCE TO RELATED APPLICATION

This application claims the benefit of U.S. Provisional Patent Application No. 63/507,047, filed Jun. 8, 2024, which is incorporated herein by reference in its entirety.

### STATEMENT REGARDING FEDERALLY SPONSORED RESEARCH

This invention was made with government support under CA175008 awarded by the National Institutes of Health. The government has certain rights in the invention.

### BACKGROUND

Patients with cancer may be subject to serial sampling of their cancerous cells and tumors. Such sampling may include genetic sequencing (e.g., serial bone marrow aspirates or peripheral blood samples tested with next generation sequencing (NGS) targeted panels, serial “liquid biopsies” sequencing circulating tumor DNA of solid tumors, or serial sampling of the solid tumor tissue itself).

Cancer relapses may result from genomic and epigenomic evolution of malignant clones due to genetic drift (random changes in allele frequencies over generations) and selection pressure exerted by sequential cancer treatments. This may cause subclones that are better adapted to reproduce in the context of each treatment to have a survival advantage over other subclones. Thus, the subclones that are vulnerable to a particular treatment are reduced or eliminated. Conversely, subclones that harbor pre-existing resistance mutations or that evolve mechanisms of resistance may then expand and come to dominate at the time of disease progression or relapse, thereby generating the malignant clones. This cancer response may model Darwinian selection, much like antibiotic and pesticide resistance.

As such, technologies which address the clonal evolution that occurs under conventional cancer treatments are needed.

### BRIEF DESCRIPTION OF THE DRAWINGS

Detailed descriptions of implementations of the present invention will be described and explained through the use of the accompanying drawings.

FIG. 1 is a block diagram showing some of the components typically incorporated in at least some of the computer systems and other devices on which the disclosed system operates in accordance with some embodiments of the present technology.

FIG. 2 is a system diagram illustrating an example of a computing environment in which the disclosed system operates in some embodiments of the present technology.

FIG. 3 is a schematic illustrating a workflow by which how a computing system ingests and processes genomic and clinical data to generate visualizations, according to some implementations.

FIGS. 4A-4G illustrate an example user interface, according to some implementations.

FIG. 5 is a flowchart illustrating a process for training and using AI/ML models associated with cancer genomes, according to some implementations.

The technologies described herein will become more apparent to those skilled in the art from studying the Detailed Description in conjunction with the drawings. Embodiments or implementations describing aspects of the invention are illustrated by way of example, and the same references can indicate similar elements. While the drawings depict various implementations for the purpose of illustration, those skilled in the art will recognize that alternative implementations can be employed without departing from the principles of the present technologies. Accordingly, while specific implementations are shown in the drawings, the technology is amenable to various modifications.

### DETAILED DESCRIPTION

Visualizing kinetics of each patient's cancer clones and subclones, aligned with swimmer plots depicting the different treatments and the responses (e.g., tumor burden as assessed by pathology, flow cytometry, tumor blood marker like CEA or CA-125, and/or imaging) may be useful to guide the choice of therapy.

There are no existing technologies to model cancer evolution in a manner that facilitates treatment decisions. Conventional systems offer visualizations of clinical data, but they do so without incorporation of cancer genomics. Some systems may focus on developing “cancer digital twins” to predict drug sensitivity, but they may not be rooted in evolutionary theory and may rely on characterization of tumors with methods like single-cell RNA sequencing, advanced flow cytometry (like CyTOF), proteomics, and/or functional or digital-surrogate drug assays that may not (1) be clinically available or (2) possess rapid enough turn-around time to be useful in a clinical setting.

The present technology comprises a system that addresses the pitfalls of conventional cancer treatments, that is, by focusing on the genomic evolution of cancer cells occurring under selective pressure from cancer treatments. The genomic evolution may include epigenomic and “ecosystem” (e.g., immunologic) evolution.

### Clone Identification and Tracking System

The present technology comprises a system having a platform-agnostic solution for identifying and tracking clones within cancer cell populations, and for analyzing these clones alongside related clinical data. The system identifies clonal evolution patterns (e.g., genetic diversification, clonal expansion, clonal selection) of a cancer based on genetic and or molecular changes of the cells, such as a cancer cell or the immune cells which comprise of the tissues that are analyzed in a given sample tested. The system may be used with bulk cell sequencing (e.g., bulk tumor cell sequencing) or with single cell sequencing methods and data. The system workflow comprises accessing a nucleotide sequencing file (e.g., Binary Alignment Map (BAM) or variant call file (VCF) sequencing files), filtering the VCFs to minimize false positive results, annotating the VCFs to a customized VCF format, using an algorithm to cluster variants (e.g., PyClone-VI) and then an algorithm to infer the phylogenetic trees (e.g., Pairtree, a Markov Chain Monte Carlo clustering method). The present technology further comprises systems incorporating phylogenetics, such as correlating longitudinal phylogenetics with clinical data and biomarkers. The systems of the present technology may produce dynamic, web-based visualizations that may be useful to support clinicians and patients in their choices of sequential cancer treatments.

The visualization can include one or more of a swimmer plot of relevant clinical events, one or more line graphs of relevant continuous laboratory parameters, fishplots of clonal evolution, a sortable table of specific genes and their associated clone number, or a phylogenetic tree. Hovering over many of the components reveals more detailed information. Separated “shapes” that underlie the fishplot and correspond to contraction/expansion of individual clones may be presented on a separate browser tab. Applicable to monitoring patients after allogeneic hematopoieitic cell transplantation, clones that derive from donor cells will be highlighted in the fishplots and/or indicated by boxes around the relevant nodes in the phylogenetic trees.

The present technology may be interoperable with various data standards, including those most commonly employed in electronic medical records (EMRs) or electronic health records (EHRs). In some embodiments, the system is made interoperable with institutional and industry-standard research databases as well (e.g., genetic databases). In some embodiments, the present technology employs the NIH Global Patient Identifier to track patients. This allows clinical data, research data, and next-generation sequencing data to be accessible (i.e., accessible from a lab or clinic or hospital).

In some embodiments, the cancer referred to in the present technology comprises a solid tumor or a hematologic cancer. Additional nonlimiting examples of cancer include a breast cancer, a lung cancer, a colorectal cancer, a prostate cancer, a skin cancer, a bladder cancer, an ovarian cancer, a pancreatic cancer, a leukemia, a non-Hodgkin lymphoma, a kidney cancer, a liver cancer, a thyroid cancer, a brain cancer, a stomach cancer, an esophageal cancer, a cervical cancer, a uterine cancer, a testicular cancer, or a multiple myeloma.

### Data Usage

In some embodiments, the system or any component thereof operates in the absence of manual data entries. In some embodiments, the system or any component thereof may use data that is transferred from one or more application programing interfaces (APIs), for example, via a data table. The system may comprise the use of data that was gathered during research studies, such as drug sensitivity data, or clinical trial data. In some implementations, the system uses custom disease-specific whitelists of genetic variants that are true positives and sequencing platform-specific blacklists of reported genetic variants that are, in fact, false positives.

In some embodiments, the system incorporates use of a genetic dataset to identify genetic variants in a cancer cell or a population of cancer cells. The genetic dataset may comprise a genetic database (e.g., NCBI The Single Nucleotide Polymorphism database (dbSNP) or the Catalogue of Somatic Mutations in Cancer (COSMIC) database), patient data (e.g., from a clinical trial dataset or a biological sample taken from the subject, EMRs, or EHRs), or other laboratory data. The genetic variants may be useful to track malignant clones. In some embodiments, the system of the present technology identifies or incorporates data from a known pathogenic mutation or a variant of uncertain significance (VUS) or benign polymorphism.

The genetic variants may be entered into a variant call file and/or may be filtered to remove sequencing errors and/or to identify variants predicted to have clinical significance (i.e., “variant filtering”). In some embodiments, the variant filtering correlates an allele frequency with a clinical result or clonal evolutionary event (e.g., genetic diversification, a clonal expansion, or a clonal selection) to identify potential evolutionary patterns that may result from a given cancer treatment. Nonlimiting examples of cancer treatments include chemotherapy, radiation therapy, and immunotherapy.

The variant filtering may be conducted according to any variant filtering steps known in the art. For example, in some embodiments, the variant must meet an allele frequency threshold at one or more time points to be considered. This may be assessed using the following threshold: (alternate observations)/(alternate observations+reference observations)≥x %, wherein the “observations” may comprise the number of instances a specific allele is observed. The threshold x is set according to NGS platform-specific and genetic locus-specific parameters.

In some embodiments, an allele fraction outlier p-value must be less than 1% at one or more timepoints. The allele fraction outlier p-value output by an Archer analysis may be the probability that the variant is due to background noise. Any variants may be removed with simple strand bias (e.g., p-value of ≤0.05). In some embodiments, only variants on both autosomes and sex chromosomes are used.

The present technology may further comprise a user interface that enables selection of a patent identifier, a time range (which alters the x-axis of the charts shown on the right), specific dates, or various plots. The charts themselves may be interactive. The charts may display the patient information, how long the patient was followed for, the dates of their last follow-up, dates when an event occurred, and biomarker information (including date of measurement, and measurement data). The charts may display various allele frequencies for different mutations and VUSs.

When utilizing data in the system, one or more of the following modeling assumptions may be made: (1) samples are diploid, (2) minor and major copy number of the segment overlapping the mutation are each 1, (. (3) in infinite sites assumption is that each mutation occurs only once, and (4) the infinite alleles assumption is that mutations do not revert back to a different allele.

The system may use and/or analyze data using one or more of data from a solid tumor (e.g., a biopsy or circulating tumor DNA).

The present technology may comprise one or more components (e.g., computing devices) for configuring or storing data. Nonlimiting examples of such components include the use of one or more of (a)-(j):
(a) a microservice configuration (e.g., AWS Elastic Container Service),
(b) a pipeline automation tool (e.g., Nextflow) for the pipeline in the release build,
(c) a pipeline automation tool input (e.g., Nextflow) from BAM and VCF files,
(d) a pipeline automation tool input (e.g., Nextflow) from a clinical database or spreadsheet of clinical data input,
(e) a log data management tool (e.g., AWS CloudWatch) for logs analysis,
(f) an engine for connecting with third-party systems (e.g. Application Programming Interface),
(g) units to write and/or run integration tests on the platform's backend and frontend,
(h) security solutions to launch data environments in a logically isolated virtual network,
(i) a web-based service data storage system, or
(j) a long-term storage system.

The microservices may act as a software language programming application to assist in writing, editing, and compiling code with its own database. In some embodiments, the microservices communicate through HTTP requests and/or an Advanced Message Queuing Protocol (AMQP), with a message-broker software for the AMQP protocol. Web-based Visualizations

The user interface of the present technology may comprise a web-based visualization for visualizing kinetics of cancer clones and subclones, aligned with swimmer plots depicting different cancer treatments and responses (e.g., tumor burden as assessed by pathology, flow cytometry, tumor blood marker like CEA or CA-125, and/or imaging). This may be useful to guide the choice of treatment and/or predict clonal evolution (e.g., clonal evolution in response to a cancer treatment).

In some embodiments, the web-based visualizations contain a swimmer plot of relevant clinical events. This may comprise one or more line graphs of relevant continuous laboratory parameters, time courses that show changes in clonal architecture in cancer/tumor evolution (e.g., fishplots of the clonal evolution), and/or a sortable table of specific genes and their associated clone number. In some embodiments, the visualizations comprise a phylogenetic tree. The components of the phylogenetic tree may reveal more detailed information on the clonal evolution and genomic relationship to other clones.

The fishplots may display information of a clone prevalence a clonal phylogeny. The fishplots may further comprise separated “shapes,” formed by Bezier splines in some implementations, that correspond to contraction/expansion of individual clones. This may be presented on a separate browser tab. Applicable to monitoring patients after allogeneic hematopoietic cell transplantation, clones that derive from donor cells may be noted in the fishplots and/or indicated by boxes around the relevant nodes in the phylogenetic trees.

In some embodiments, the web-based visualization comprises a graphical user interface (GUI). The GUI may permit a user to interact with data from the system, such as, by navigating through a database, a table, or other data forms. The GUI may enable the user to (1) view clinical information (e.g., dose or route-of-administration of a treatment used), (2) visualize clonal evolution (e.g., via fishplots and phylogenic trees), or (3) sort data by clones to identify mutations or variants of interest.

In some embodiments, the GUI allows a user to consult with a clinical professional (e.g., a molecular oncology expert).

### Additional Uses

The present technology may be useful in one or more of (1) developing cancer treatment strategies that account for tumor evolution; (2) informing or planning personalized treatment decisions for treating cancer; (3) demonstrating or predicting how cancer treatments impact cancer overtime, (4) planning a clinical trial, (5) predict future evolution of a clone or cancer cell, (6) predict optimal treatments for a subject in need thereof, (6) transfer learning between phylogenetic trees and machine learning (ML) and artificial intelligence (AI)-based models. One or more of (1)-(5) may be Al or ML implemented or assisted.

In some embodiments, the present technology comprises a method of identifying or predicting evolution of a cancer cell or a method of selecting a treatment for a subject with cancer to reduce, prevent, or otherwise ameliorate cancer cell evolution. The methods may comprise (i) obtaining a genetic dataset derived from the cancer cell; and (ii) applying, to the genetic dataset, a non-transitory medium with instructions stored thereon that, when executed by a processer of a computing device, causes the computing device to perform steps for (a) identifying a pathogenic mutation or a VUS in the genetic dataset derived from the cancer cell; (b) correlating the pathogenic mutation or VUS with one or more of a genetic diversification, a clonal expansion, or a clonal selection; and (c) displaying, on a user interface, a kinetic property of the cancer cell population or a predicted or known response of the cancer cell population to a cancer treatment.

One or more of these steps may comprise any feature of the present technology. For example, the non-transitory medium of the methods may further comprise one or more of a microservice configuration, a pipeline automation tool, a pipeline automation tool input, data management tool, an engine for connecting with third-party systems, a unit to write or run integration tests on a backend or a frontend of the non-transitory medium, or data storage system.

While the illustrative visualization have been developed using acute myeloid leukemia (AML) as an exemplary cancer, one of skill in the art would understand that the systems and methods of the present technology are applicable to any cancer. The disclosed systems and methods can be used with bulk tumor sequencing or with single cell sequencing and are designed to be platform-agnostic.

### Computing Environment

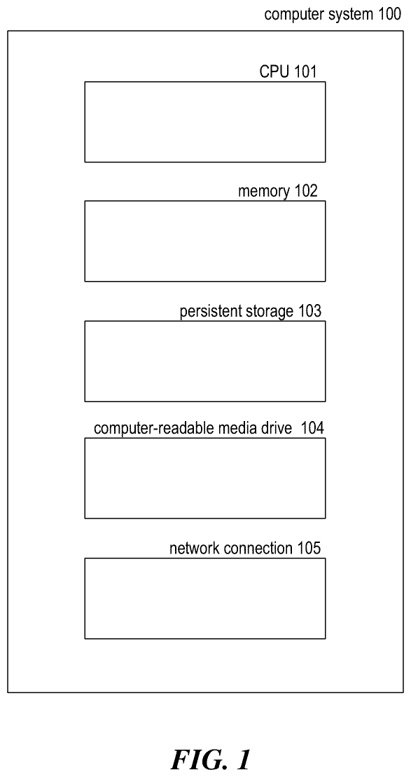

FIG. 1 is a block diagram showing some of the components typically incorporated in at least some of the computer systems (“computer system 100”) and other devices on which the system of the present technology operates. In various embodiments, these computer systems and other devices may include server computer systems, desktop computer systems, laptop computer systems, netbooks, mobile phones, personal digital assistants, televisions, etc. In some embodiments, the computer systems and devices include zero or more of each of the following: a central processing unit (CPU) (“CPU 101”) for executing computer programs; a computer memory (“memory 102”) for storing programs and data while they are being used, including the facility and associated data, an operating system including a kernel, and device drivers; a persistent storage device (“persistent storage 103”), such as a hard drive or flash drive for persistently storing programs and data; computer-readable media drives (“computer-readable media drive 104”) that are tangible storage means that do not include a transitory, propagating signal, such as a floppy, CD-ROM, or DVD drive, for reading programs and data stored on a computer-readable medium; and a network connection (“network connection 105”) for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

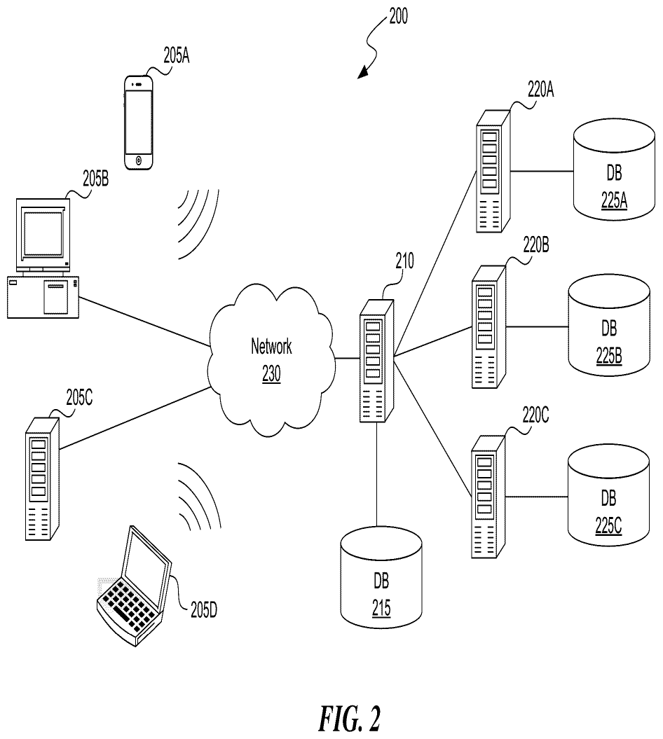

FIG. 2 is a system diagram illustrating an example of a computing environment (“200”) in which the system of the present technology may operate. In some embodiments, environment 200 includes one or more client computing devices (“205A-D”), examples of which can host the computer system 100 of FIG. 1. Client computing devices 205A-D operate in a networked environment using logical connections through network (“230”) to one or more remote computers, such as a server computing device.

In some embodiments, the server (“210”) is an edge server which receives client requests and coordinates fulfillment of those requests through other servers (“220A-C”), such as servers 220A-C. In some embodiments, server computing devices 210 and 220A-C comprise computing systems, such as the system 100 of FIG. 1. Though each server computing device 210 and 220A-C is displayed logically as a single server, server computing devices can each be a distributed computing environment encompassing multiple computing devices located at the same or at geographically disparate physical locations. In some embodiments, each server 220A-C corresponds to a group of servers.

Client computing devices 205A-D and server computing devices 210 and 220A-C may each act as a server or client to other server or client devices. In some embodiments, servers 210 and 220A-C connect to a corresponding database (“215,” “225A-C”). Each server 220A-C may correspond to a group of servers, and each of these servers can share a database or can have its own database. Databases 215 and 225A-C warehouse (e.g., store) information such as genome data, clinical data, phylogenetic trees, or intermediate or processed data structures generated based on other data. Though databases 215 and 225A-C may be displayed logically as single units, databases 215 and 225A-C may each be a distributed computing environment encompassing multiple computing devices, may be located within their corresponding server, or may be located at the same or at geographically disparate physical locations.

Network 230 may be a local area network (LAN) or a wide area network (WAN), but may also be other wired or wireless networks. In some embodiments, network 230 is the Internet or some other public or private network. Client computing devices 205A-D are connected to network 230 through a network interface, such as by wired or wireless communication. While the connections between server 210 and servers 220A-C are shown as separate connections, these connections may be any kind of local, wide area, wired, or wireless network, including network 230 or a separate public or private network.

### Processing Genomic and Clinic Data

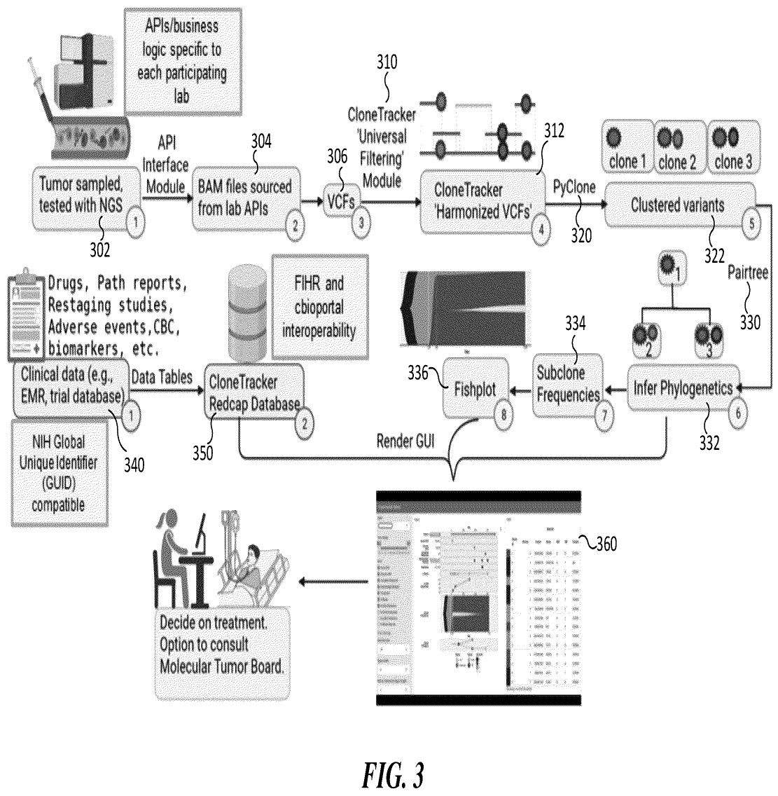

FIG. 3 is a schematic illustrating a workflow by which how a computing system ingests and processes genomic and clinical data to generate visualizations, according to some implementations. Aspects of the process shown in FIG. 3 can be performed by a computer system, such as the computer system 100, or in a HIPAA-eligible cloud-based environment such as Cirro.bio that can handle applicable pipelines, manage input and output files, and automate deliver of data from lab instruments and electronic health records directly to processing scripts described herein.

As patients are treated, tumor samples 302 are collected and sequenced to detect cancer genomes that are present. The sequenced genomes can be stored in genome files 304 (e.g., Binary Alignment Map (BAM) files or Variant Call Files (VCFs)), which are ingested by the computer system from laboratory systems or databases from the laboratories that collect and sequence the tumor samples 302. Each laboratory that collects tumor samples may have different business processes that dictate different data formats or that cause the genome files to contain different information. Furthermore, each laboratory may use different application programming interfaces (APIs) by which the computer system can ingest the genome files 304. Accordingly, the system may ingest genome files 304 in a variety of different formats, via a variety of different APIs.

The computer system converts the ingested genome files 304 to corresponding text files 306, configured for example as a Variant Call Format (VCF) file that stores gene sequence variations as text (e.g., .txt or .tsv).

The computer system then applies a filtering module 310 to filter the text files 306. The output of filtering is a set of filtered text files 312. In some implementations, the filtering module 310 filters text files 306 based on identifying unique chromosome and position combinations in each file 306. The filtering module 310 can loop over rows in the text file that contain the same unique position and determine whether these rows satisfy criteria for including or excluding the values they contain. For example, the filtering module 310 uses a disease-specific allow list and a NGS platform-specific deny list that contain, respectively, gene variants that are to be included or discarded. In still other examples, the filtering module 310 determines whether a chromosome has an identifier of a known mutation, for example an identifier in the Single Nucleotide Polymorphism Database (dbSNP) maintained by the United States National Institutes of Health, or an identifier in the Catalog of Somatic Mutations in Cancer (COSMIC). The filtering module 310 can further remove potential artifacts or entries that are variations of the mutations in the deny list. In some implementations, the filtering module 310 retains benign variants and/or single nucleotide polymorphisms (SNPs) in the filtered file, as well damaging variants that are detected in the ingested files 304.

Once unique variants have been identified from a text file 306, the filtering module 310 can calculate an allele frequency for each unique variant. The filtered file 312 can then be stored in a database.

In some implementations, an unfiltered text file 306 is received for each sample or clinical measurement timepoint for a given patient. After filtering, the filtered text files 312 that correspond to the same patient can be combined into one file. To generate a patient-specific file, the filtering module 310 can iterate over a data frame that includes patient identifiers and associated sample data (e.g., sources of samples, time and date sample was taken, sample identification, and/or tissue or sample type). For each patient, the filtering module 310 combines any detected variants into a single data frame and applies one or more filters to this combined data frame, such as removing variants that do not have dbSNP ID or COSMIC ID, removing likely sequencing artifacts, and removing variants that are on the deny list. The filtering module 310 can also remove any variants that do not meet an allele frequency threshold (e.g., 5%). The filtered data is written to the single patient-specific file, which can specify the number of unique filtered variants for the patient.

The computer system processes the filtered text files 312 using a clonal population structure generator 320 (e.g., PyClone-VI). The output of the clonal population structure generator is a set of clustered variants 322. PyClone-VI uses a Bayesian hierarchical model to infer clones. The core idea is to model the observed variant allele frequencies as mixtures of distributions corresponding to different clonal populations within the tumor. Initial parameters for clonal frequency distributions are set. The model iterates to optimize the likelihood of the data, given the model parameters, by sampling from the posterior distribution using Markov Chain Monte Carlo (MCMC) methods. It ensures that the MCMC chains have converged, indicating that the model has adequately explored the parameter space.

The clustered variants 322 are next processed through an evolutionary history generator 330 (e.g., Pairtree) that reconstructs changes in cancer genomes over time.

The computer system also ingests and processes clinical data associated with cancer patients. Clinical data can include a series of clinical events associated with a patient. These clinical events can include, for example, measurements of data such as, pathology, cytogenetic, flow cytometric, or lab data, wherein the clinical data can include the value of the data measured and the time the measurement was taken. Clinical events can also include categorical data, such as any treatments applied to a patient, such as receiving a transplant; or outcomes related to treatments, such as cancer remission status or the patient contracting infection or chronic graft-versus-host disease after receiving a transplant. In some implementations, the clinical data can further include status of the patient, such as whether the patient is deceased.

The computer system retrieves clinical data from clinical data sources 340, such as electronic medical records (EMRs) or databases associated with clinical trials. The clinical data is processed and stored in a database 350 associated with the computer system, where standard data records can be generated based on the clinical data received from different sources. Data can be retrieved from electronic medical records or clinical trial databases automatically over applicable APIs, without requiring manual data entry. Additionally, the database 350 can be compatible with common clinical trial databases, in some implementations. In some implementations, the computer system uses a large language model (LLM) to read clinical data from electronic medical records and to generate a standardized representation of the clinical data. When processing the clinical data through the LLM, the LLM can deduplicate patient data and deidentify patient data such that the records in the computer system's database 350 are anonymized.

Based on the filtered text files 312, the set of clustered variants 322, the output of the evolutionary history generator 330, and/or the clinical data records, the computer system generates data structures that can be used to generate one or more visualizations within a user interface 360. These data structures can include, for example, a phylogenetic tree 332 in which clones (clustered variants) are represented as nodes in the tree and branches identify relationships between the clones, such as one clone arising from another clone. Another example data structure is a subclone frequency data structure 334, which represents the frequency of a given clone or of an individual gene variant within a patient sample. Based on the subclone frequency data structure 334, the computer system can also generate a fishplot data structure 336, which identifies the prevalence of clones or individual gene variants at different times. Data structures can also be generated to represent the clinical data. For example, a clinical event data structure can be generated that includes a measurement from a patient, the date the measurement was taken, and the status of the patient at the measurement. The clinical event data structure can also include a description of each clinical event that can be displayed when a user interacts with a visualization of the clinical event data. These clinical event data structures can be combined into a data frame that organizes events in a desired order, such as in increasing date order. Other events can be ordered based on other desired factors. For example, events associated with chronic graft-vs-host disease can be organized based on severity of the disease, from mild, to moderate, to severe.

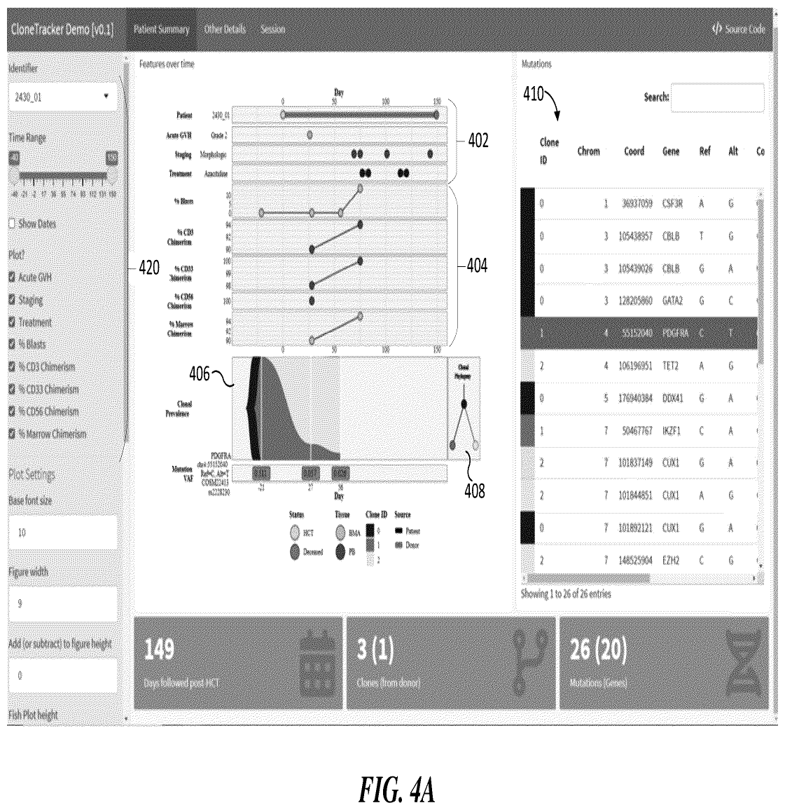

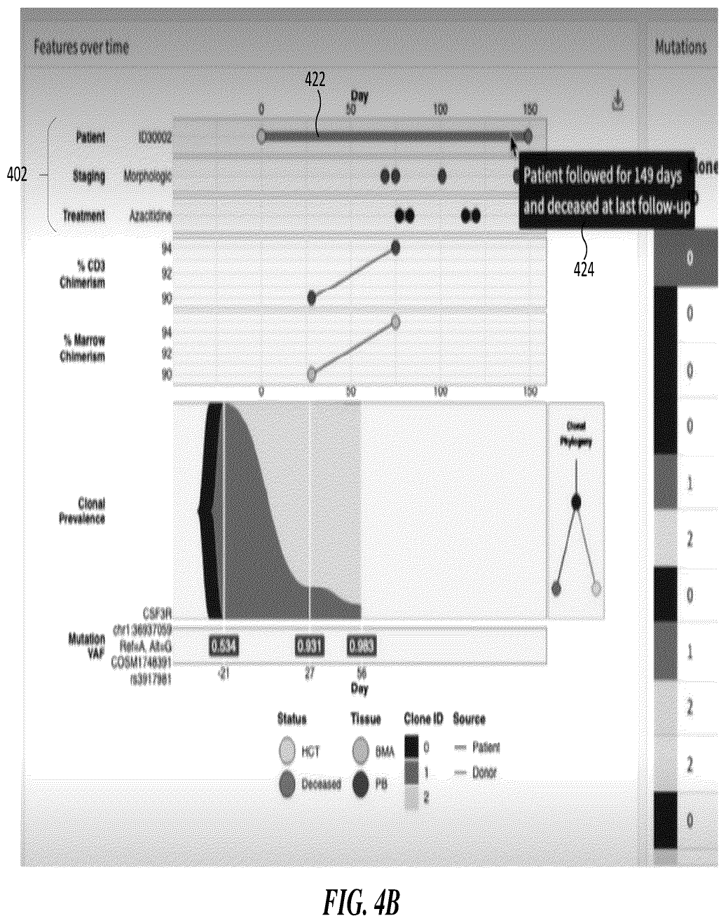

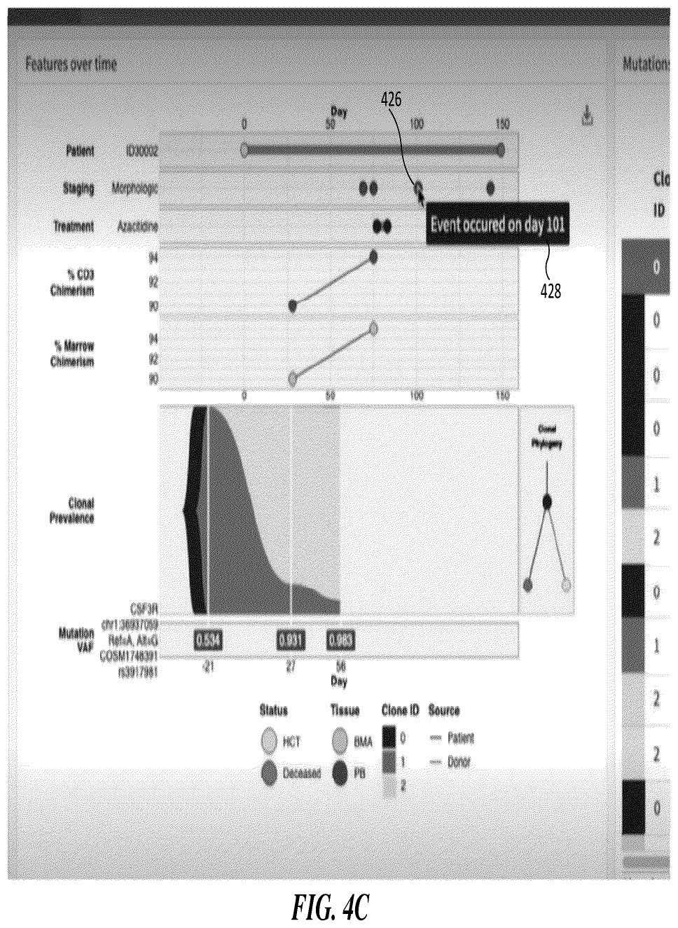

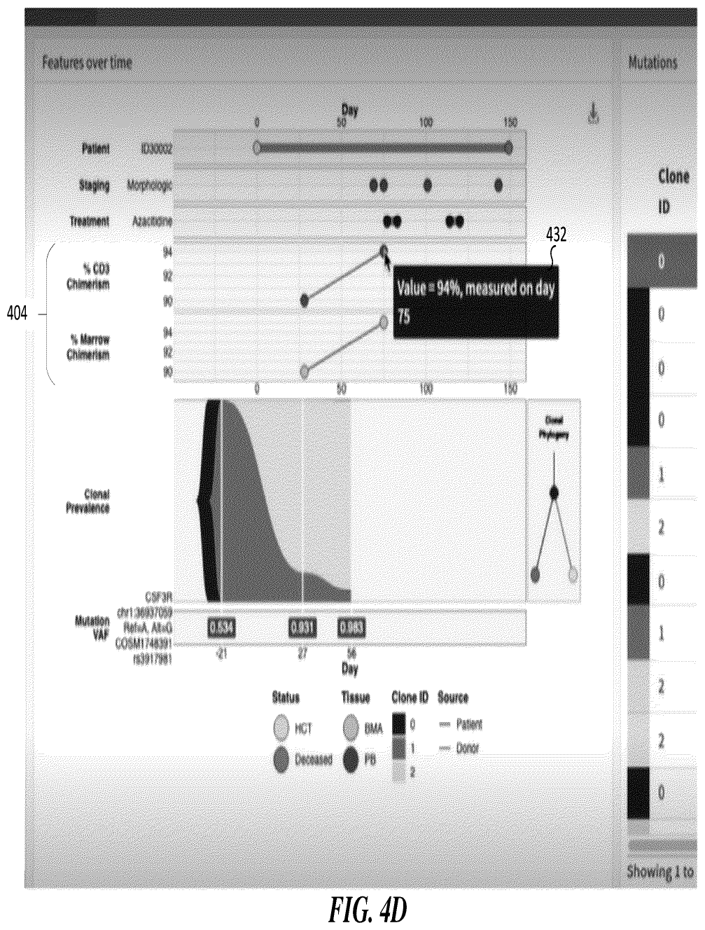

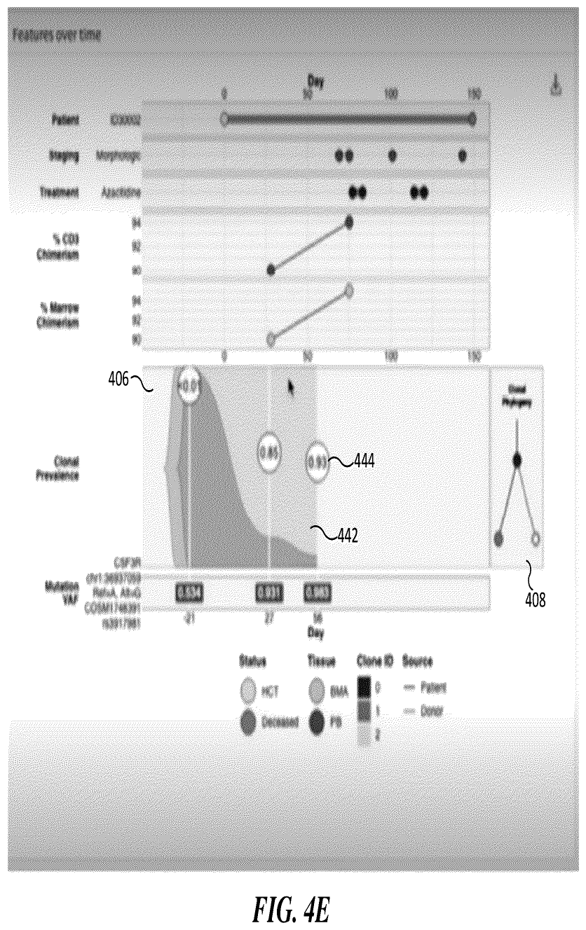

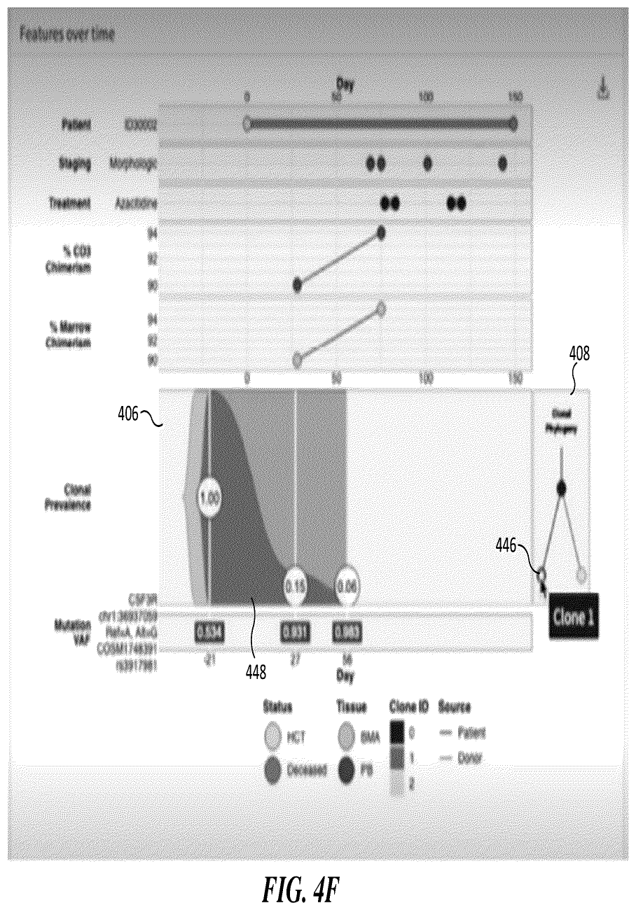

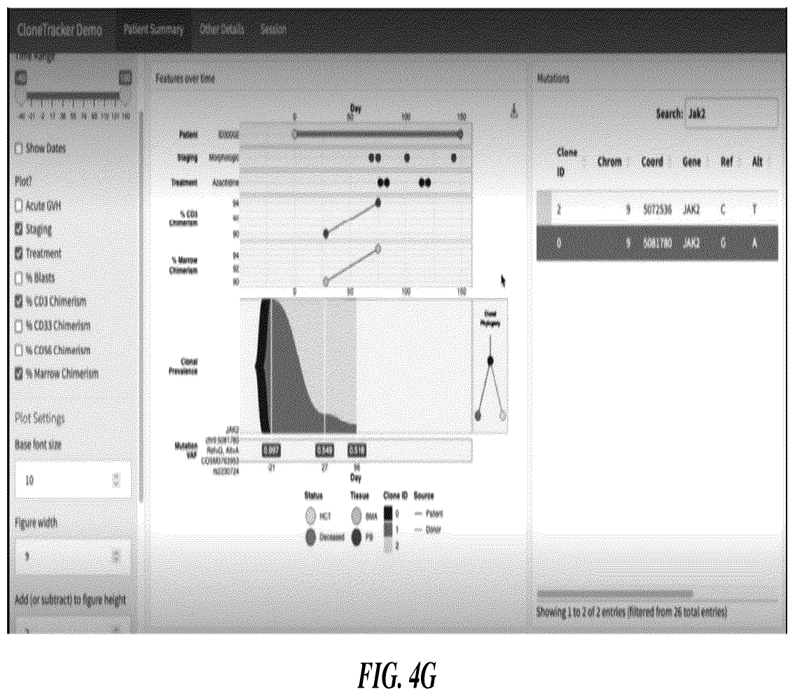

Based on the clinical data records in the database 350 and the processed genome data structures, the computer system generates a user interface 360 with one or more visualizations of the data structures and their correlations. FIGS. 4A-4G illustrate an example of the user-interface 360 generated based on the process shown in FIG. 3. The user interface 360 can be generated by the computer system and rendered for display by a user device, such as client computing devices 205A-D in FIG. 2.

FIG. 4A illustrates a user interface 360 as a dashboard with multiple interactive visualizations of cancer genomic data and associated clinical data. The example in FIG. 4A shows a donor-derived, PDGFRA mutation at 51% variant allele frequency initially in a patient, and 0.026% VAF by day 56. In this example, there was one predominant donor-derived clone post-transplant with variants in TET2 and EZH2 among others. Both donor and patients shared some variant of uncertain significance (VUSs) in CSF3R, CBLB, GATRA2, and DDX41, as indicated by the hypothetical (fantastical) ‘parental’ clone 0 that is the variants shared between patient and donor. The hypothetical parental clone shared across people may only be relevant to the allotransplant setting.

The dashboard includes various visualizations, such as a swimmer plot 402 of relevant clinical events, a line graph 404 of relevant continuous laboratory parameters, a fishplot 406 of clonal evolution, a phylogenetic tree 408 depicting evolution of a genome, and a sortable table 410 of specific genes and their associated clone number. The swimmer plot 402 and line graph 404 can be generated based on the clinical event data structures. For example, the swimmer plot 402 represents different event types in each “lane” within the plot, where the relevant events for each event type are organized in order of time and respectively represented by an element (e.g., a point) displayed on the plot 402. The line graph 404 represents any parameters of a patient that were measured at each of a plurality of measurement times by corresponding points on the graph 404. The fishplot 406 can be generated based on the fishplot data structure, while the phylogenetic tree 408 can be generated based on the phylogenetic tree data structure. In some implementations, some or all of the visualizations can be displayed with the same x-axis, such that the relevant data in the visualization is plotted against the same interval of time. A user can therefore readily visualize how a patient's treatment proceeds over time.

The dashboard can include a set of filters 420 that can be selected by a user. These filters can include, for example, specifying an identifier of the patient whose data should be plotted in the visualizations, specifying time range or specific dates for the displayed patient data, or specifying the particular events or parameters that should be displayed. When the time range filter is modified, for example, each of the visualizations that include data plotted against time can be updated to plot the applicable data within the modified time range.

Some or all of the visualizations in the user interface 360 can be interactive for a user to view additional information about the underlying data. When a user interaction with an element in the visualizations is detected, information associated with the element can be displayed within the user interface 360. User interactions can include hovering a cursor over elements, selecting (e.g., clicking on or tapping) elements, speaking an identifier of an element into a voice input interface, looking at an element with a gaze tracking interface, or the like. The information that is displayed can be identified based on genomic data structures or clinical event data structures associated with the visualizations.

FIG. 4B, for example, illustrates an interaction with the swimmer plot 402, where a cursor is positioned over an element 422 in the plot 402. When the cursor is positioned over the element 422, a pop-up description or tooltip 424 is displayed with additional information associated with the element, such as patient information, how long the patient was followed, date of last follow-up, any dates when events occurred, biomarker information, or current status of a patient. In some implementations, the description in the tooltip 424 is generated based on a template associated with each type of element, and is stored in a clinical event data structure from which the element was generated. For example, the description in the tooltip 424 shown in FIG. 4B can be generated based on the following template: “Patient followed for <x> days and <status> at last follow-up,” where <x> is a variable corresponding to the number of days the patient was followed, and <status> is a variable for the patient's current status (e.g., “deceased,” “alive,” “in remission,” etc.). When generating the visualization or rendering the tooltip, the computer system can retrieve values to populate the variables in the template from a data structure associated with the visualization. Other implementations generate tooltip descriptions in other ways. For example, the LLM can generate tooltip descriptions and enter them in the data structure that populates the tooltip display.

FIG. 4C illustrates another example interaction with the swimmer plot 402. In FIG. 4C, a user is hovering a cursor over an element 426 in a region of the plot that shows morphological stages of a patient's condition. When the cursor hovers over the element 426, a tooltip 428 is displayed to indicate that an event occurred on a certain day within the time period in which the patient was monitored and provides details from the pathology report, such as bone marrow cellularity, blast percentage by morphology and flow cytometry, karyotype, PCR, and FISH results. Where relevant, in some implementations, key information from imaging (e.g., CT scan, PET scan, MRI, ultrasound, X-ray) reports may be displayed.

FIG. 4D illustrates an example interaction with the line graph 404 of measured laboratory parameters. When a user hovers over or selects a data point on the line graph 404, a tooltip 432 can be displayed to indicate the value of the parameter plotted by the data point, the unit of measurement, and the day on which the parameter's measurement was taken. In some implementations, the normal range for the lab test can be included in the tooltip.

FIGS. 4E-4F illustrate example interactions with the fishplot 406 and phylogenetic tree 408. The fishplot 406 illustrates clonal evolution over time, for example by representing percentages of each of the clones within patient samples on the vertical axis and representing time on the horizontal axis. In FIG. 4E, hovering over a portion of the fishplot 406 causes the visualization to display information about clone prevalence. For example, the selected clone prevalence 442 is highlighted in FIG. 4E while prevalence of other clones is greyed out, and numerical values 444 indicating the prevalence of the selected clone are added to the plot 406.

In some implementations, the fishplot 406 is linked with the phylogenetic tree 408. As shown in FIG. 4F, a cursor hovering over an element 446 in the phylogenetic tree 408 that represents a given clone causes the fishplot 408 to display or emphasize information about the corresponding prevalence of the clone 448.

The visualizations generated by the computer system can further represent allele frequency for different mutations. FIG. 4G illustrates that when a particular gene variant is clicked on the table on the right, the gene name and the variant nomenclature are displayed at the bottom of the fishplot and its variant allele frequency is displayed on the horizontal bar beneath the fishplot at each sample time point. For example, displaying the gene variant nomenclature and allele frequency beneath the fishplot can enable a user to observe existence and proliferation of clones that are driving a patient's cancer to relapse. In conjunction with other visualizations on the display, the user can also observe how clinical events relate to the proliferation of these clones.

Together, the visualizations in the user interface 360 provide an intuitive display that maps clinical treatments, biomarkers, stages, and/or clonal evolution to each other. A user can interact with the visualizations to readily determine the effects of different treatments. For example, doctors and patients can use the visualizations to personalize treatment decisions for a patient, based on treatments that have or have not been successful for other similar patients. Researchers can use the visualizations to develop cancer treatment strategies that account for tumor evolution. Industry partners can use the visualizations to launch novel types of precision medicine trials and to obtain de-identified, real-world data about how prescriptions or treatments affect cancer over time.

Deriving Insights from Correlated Genomic and Clinical Data

Once genomic and clinical data has been ingested by a computer system and processed into data structures that correlate cancer genomes over time and clinical events related to these genomic histories, a computer system can use the data structures to train artificial intelligence/machine learning (AI/ML) models, instead of or in addition to generating interactive visualizations based on the data structures. These models can be used, for example, to predict future evolution of a cancer cell or to identify treatments tailored to a patient's unique genomic history.

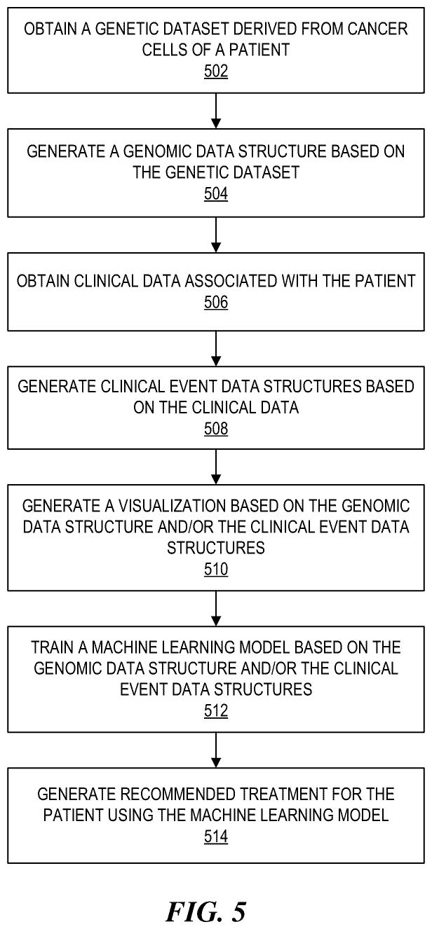

FIG. 5 is a flowchart illustrating a process 500 for training and using Al/ML models associated with cancer genomes, according to some implementations. The process 500 can be performed by a computer system, such as the computer system 100. Other implementations of the process can include additional, fewer, or different steps, or can perform the steps in different orders.

At 502, the computer system obtains a genetic dataset derived from cancer cell samples of a patient, where the genetic dataset includes cancer cell genome data measured at each of a plurality of times. A genomic data structure is generated based on the genetic dataset, at 504. For example, the genetic dataset can be obtained based on ingesting and filtering genome files 304, as described with respect to FIG. 3.

At 506, the computer system obtains clinical data associated with the patient that includes a plurality of clinical events. Clinical event data structures are generated, at 508, based on the clinical data.

At 510, the computer system can generate a visualization based on the genomic data structure and/or the clinical event data structures. The generated visualizations can include the visualizations described with respect to FIGS. 4A-4G, for example.

Similar genomic data structures and clinical event data structures can be generated for a large set of patients. At 512, the computer system uses the genomic data structures and the clinical event data structures for the patients to train a machine learning model. A “model,” as used herein, can refer to a construct that is trained using training data to make predictions or provide probabilities for new data items, whether or not the new data items were included in the training data. For example, training data for supervised learning can include items with various treatments applied to a cancer patient and a measurement of a patient parameter or cancer genomic data after the treatment has been applied. A new data item can have a treatment type that the model can use to predict a parameter or a prevalence of a certain cancer genomic variants that will follow the treatment type. Examples of models include neural networks, support vector machines, decision trees, Parzen windows, Bayes, clustering, reinforcement learning, probability distributions, decision trees, decision tree forests, and others. Models can be configured for various situations, data types, sources, and output formats.

Many machine learning techniques are based on neural networks. A neural network model has three major components: architecture, cost function, and search algorithm. The architecture defines the functional form relating the inputs to the outputs (in terms of network topology, unit connectivity, and activation functions). During a training process, a computing system performs a search in weight space for a set of weights that minimizes the objective function.

A neural network has a set of input nodes that receive input data. The input nodes can correspond to functions that receive the input and produce results. These results can be provided to one or more levels of intermediate nodes (“hidden layers”) that each produce further results based on a combination of input node results. A weighting factor is applied to the output of each input node before the result is passed to the hidden layer nodes. The hidden layer can have lower dimensionality than the input and/or output layers, in some implementations. At a final layer (“the output layer”), a set of output nodes are mapped to output data. Once the neural network is trained, application of the field values to the input and output nodes produces a latent vector at the hidden layer that represents features of the input data.

Some neural networks, known as deep neural networks, have multiple layers of intermediate nodes with different configurations, are a combination of models that receive different parts of the input and/or input from other parts of the deep neural network, or are convolutions-partially using output from previous iterations of applying the model as further input to produce results for the current input.

Using the trained model, the computer system can generate recommended treatments for a patient, at 514. For example, a data structure indicating a set of cancer variant genomes measured in the patient can be input to the model. Based on the prevalence of certain genomes, the model generates a recommended for a treatment type that is most likely to modify the prevalence in a desired way.

Training the machine learning model can include generating a digital twin and using transfer learning to generate a model for other systems or data types. In one example, the computer system uses phylogenetic trees for one type of cancer to create a digital twin that simulates evolution of genomic variations within the cancer over time. A model can be trained on this digital twin to predict the evolution of genomic variants based on the information it received from the digital twin (e.g., predicting formation of new branches in the phylogenetic tree, emergence of new genomic variations, or extinction of existing variations). Once the model has been trained, the knowledge it has gained can be transferred to another system, such as a model for predicting evolution of another cancer type.

The system can offer the predictions from a single model or from multiple models to users. In one implementation, the predictions are presented on separate browser tabs.

### CONCLUSION

The above examples are not intended to be exhaustive or to limit the technology to the precise form disclosed above. While specific examples for the technology are described above for illustrative purposes, various equivalent modifications are possible within the scope of the technology, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified to provide alternative or sub-combinations. Each of these processes or blocks may be implemented in a variety of different ways. Also, while processes or blocks are at times shown as being performed in series, these processes or blocks may instead be performed or implemented in parallel, or may be performed at different times. Further, any specific numbers noted herein are only examples: alternative embodiments may employ differing values or ranges.

The teachings of the present technology may be applied to other systems, not necessarily the system described above. The elements and acts of the various examples of the present technology can be combined to provide additional embodiments of the technology. Some alternative embodiments of the technology may include not only additional elements to those embodiments noted above, but also may include fewer elements.

These and other changes can be made to the present technology. Embodiments of the system may vary considerably in its specific implementation, while still being encompassed by the present technology. As noted above, specific terminology used when describing certain features or aspects of the technology should not be taken to imply that the terminology is being redefined herein to be restricted to any specific characteristics, features, or aspects of the technology with which that terminology is associated. In general, the terms used in the claims should not be construed to limit the technology to the specific examples disclosed in the specification, unless the Detailed Description section explicitly defines such terms. Accordingly, the actual scope of the present technology encompasses not only the illustrative examples, but also all equivalent ways of practicing or implementing the technology under the claims.

We claim:

1. A method comprising:
generating, by a computer system, a visualization that represents progression of a cancer genome, wherein generating the visualization comprises:
obtaining a genetic dataset derived from cancer cell samples of a patient, the genetic dataset including cancer cell genome data measured at each of a plurality of times;
generating a genomic data structure that represents the genetic dataset;
obtaining clinical data associated with the patient, wherein the clinical data includes a plurality of clinical events;
generating clinical event data structures to represent each of the plurality of clinical events based on the clinical data; and
causing a user interface to display the visualization with interactive elements that are populated based on the genomic data structure and the clinical event data structures;
detecting, by the computer system, a user input within the user interface that is associated with a first interactive element in the interactive elements; and
displaying, within the user interface, in response to detecting the user input, information associated with the first interactive element based on the genomic data structure or the clinical event data structures.

2. The method of claim 1:
wherein the clinical event data structures include, for each of the clinical events, a description of the corresponding clinical event;
wherein the visualization comprises a swimmer plot that represents one or more of the clinical events as respective interactive elements; and
wherein displaying information in response to the user interaction with the first interactive element comprises:
retrieving the description of the clinical event associated with the first interactive element from a corresponding clinical event data structure; and
displaying a tooltip containing the retrieved description.

3. The method of claim 1:
wherein the clinical event data structures include, for each of a plurality of laboratory measurement events, a value of a measurement taken at each of the laboratory measurement events;
wherein the visualization comprises a line graph that represents one or more laboratory measurement events as respective interactive elements; and
wherein displaying information in response to the user interaction with the first interactive element comprises:
retrieving the value of the measurement associated with the first interactive element from a corresponding clinical event data structure; and
displaying a tooltip containing the retrieved value.

4. The method of claim 1:
wherein the clinical event data structures include:
for each of the clinical events, a description of the corresponding clinical event; and
for each of a plurality of laboratory measurement events, a value of a measurement taken at each of the laboratory measurement events;
wherein the visualization comprises a swimmer plot that represents one or more of the clinical events within a specified period of time as respective interactive elements; and
wherein the visualization comprises a line graph that represents one or more laboratory measurement events in the specified period of time as respective interactive elements.

5. The method of claim 1:
wherein the genomic data structure includes prevalence of each of a plurality of variants within the cancer cell genome data measured at each of the plurality of times;
wherein the visualization comprises a fishplot that represents prevalence of each of the plurality of variants across a specified time period; and
wherein displaying information in response to the user interaction with the first interactive element comprises:
displaying, on the fishplot, a value of the prevalence of a first variant corresponding to the first interactive element.

6. The method of claim 5:
wherein the visualization further comprises a phylogenetic tree that represents an evolutionary relationship between each of the plurality of variants and in which the plurality of variants are represented as respective interactive elements; and
wherein the user interaction with the first interactive element comprises a selection of the first variant from the phylogenetic tree.

7. The method of claim 5:
wherein the visualization further comprises a table that lists gene variants detected in the genetic dataset;
wherein the user interaction comprises a selection of a first gene variant from the table; and
wherein displaying information in response to the user interaction with the first interactive element comprises:
displaying, on the fishplot, variant nomenclature of the first gene variant and allele frequency of the first gene variant.

8. The method of claim 1, wherein generating the visualization comprises:
generating two or more visualizations from:
a swimmer plot that represents one or more of the clinical events as respective interactive elements across a first specified time period;
a line graph that represents one or more laboratory measurement events as respective interactive elements across the first specified time period; or
a fishplot that represents prevalence of each of a plurality of variants within the cancer cell genome data across the first specified time period;
receiving a user input to modify the first specified time period to a second specified time period; and
modifying the two or more visualizations to represent corresponding clinical events, laboratory measurement events, or prevalence of each of the plurality of variants during the second specified time period.

9. The method of claim 1, wherein obtaining the genetic dataset comprises:
ingesting a plurality of genome files in non-standard formats; and
filtering the plurality of ingested genome files to generate a filtered genome file that includes the cancer cell genome data measured at each of the plurality of times.

10. The method of claim 1, wherein obtaining the clinical data associated with the patient comprises:
ingesting electronic medical records associated with the patient; and
processing the ingested electronic medical records using a large language model (LLM) to de-duplicate redundant entries and to deidentiy the patient.

11. The method of claim 1, wherein the patient is a first patient, and wherein the method further comprises:
obtaining a plurality of genetic datasets derived from cancer cell samples of a plurality of other patients;
obtaining a plurality of clinical datasets associated with the plurality of other patients;
training a model based on the plurality of genetic datasets and the plurality of clinical datasets; and
applying the trained model to the genetic dataset of the first patient and the clinical data associated with the first patient to generate a recommended treatment for the first patient.

12. A non-transitory computer readable storage medium storing executable computer program instructions, the computer program instructions when executed by one or more processors of a system causing the system to:
generate a visualization that represents progression of a cancer genome, wherein generating the visualization comprises:
obtaining a genetic dataset derived from cancer cell samples of a patient, the genetic dataset including cancer cell genome data measured at each of a plurality of times;
generating a genomic data structure that represents the genetic dataset;
obtaining clinical data associated with the patient, wherein the clinical data includes a plurality of clinical events;
generating clinical event data structures to represent each of the plurality of clinical events based on the clinical data; and
causing a user interface to display the visualization with interactive elements that are populated based on the genomic data structure and the clinical event data structures;
detect a user input within the user interface that is associated with a first interactive element in the interactive elements; and
display, within the user interface, in response to detecting the user input, information associated with the first interactive element based on the genomic data structure or the clinical event data structures.

13. The non-transitory computer readable storage medium of claim 12:
wherein the clinical event data structures include, for each of the clinical events, a description of the corresponding clinical event;
wherein the visualization comprises a swimmer plot that represents one or more of the clinical events as respective interactive elements; and
wherein displaying information in response to the user interaction with the first interactive element comprises:
retrieving the description of the clinical event associated with the first interactive element from a corresponding clinical event data structure; and
displaying a tooltip containing the retrieved description.

14. The non-transitory computer readable storage medium of claim 12:
wherein the genomic data structure includes prevalence of each of a plurality of variants within the cancer cell genome data measured at each of the plurality of times;
wherein the visualization comprises a fishplot that represents prevalence of each of the plurality of variants across a specified time period; and
wherein displaying information in response to the user interaction with the first interactive element comprises:
displaying, on the fishplot, a value of the prevalence of a first variant corresponding to the first interactive element.

15. The non-transitory computer readable storage medium of claim 12, wherein the patient is a first patient, and wherein the instructions when executed by the one or more processors further cause the system to:
obtain a plurality of genetic datasets derived from cancer cell samples of a plurality of other patients;
obtain a plurality of clinical datasets associated with the plurality of other patients;
train a model based on the plurality of genetic datasets and the plurality of clinical datasets; and
apply the trained model to the genetic dataset of the first patient and the clinical data associated with the first patient to generate a recommended treatment for the first patient.

16. A system comprising:
one or more processors; and
one or more non-transitory computer readable storage media storing executable computer program instructions, the computer program instructions when executed by the one or more processors causing the system to:
generate a visualization that represents progression of a cancer genome, wherein generating the visualization comprises:
obtaining a genetic dataset derived from cancer cell samples of a patient, the genetic dataset including cancer cell genome data measured at each of a plurality of times;
generating a genomic data structure that represents the genetic dataset;
obtaining clinical data associated with the patient, wherein the clinical data includes a plurality of clinical events;
generating clinical event data structures to represent each of the plurality of clinical events based on the clinical data; and
causing a user interface to display the visualization with interactive elements that are populated based on the genomic data structure and the clinical event data structures;
detect a user input within the user interface that is associated with a first interactive element in the interactive elements; and
display, within the user interface, in response to detecting the user input, information associated with the first interactive element based on the genomic data structure or the clinical event data structures.

17. The system of claim 16:
wherein the clinical event data structures include, for each of the clinical events, a description of the corresponding clinical event;
wherein the visualization comprises a swimmer plot that represents one or more of the clinical events as respective interactive elements; and
wherein displaying information in response to the user interaction with the first interactive element comprises:
retrieving the description of the clinical event associated with the first interactive element from a corresponding clinical event data structure; and
displaying a tooltip containing the retrieved description.

18. The system of claim 16:
wherein the clinical event data structures include, for each of a plurality of laboratory measurement events, a value of a measurement taken at each of the laboratory measurement events;
wherein the visualization comprises a line graph that represents one or more laboratory measurement events as respective interactive elements; and
wherein displaying information in response to the user interaction with the first interactive element comprises:
retrieving the value of the measurement associated with the first interactive element from a corresponding clinical event data structure; and
displaying a tooltip containing the retrieved value.

19. The system of claim 16:
wherein the genomic data structure includes prevalence of each of a plurality of variants within the cancer cell genome data measured at each of the plurality of times;
wherein the visualization comprises a fishplot that represents prevalence of each of the plurality of variants across a specified time period; and
wherein displaying information in response to the user interaction with the first interactive element comprises:
displaying, on the fishplot, a value of the prevalence of a first variant corresponding to the first interactive element.

20. The system of claim 16, wherein obtaining the genetic dataset comprises:
ingesting a plurality of genome files in non-standard formats; and
filtering the plurality of ingested genome files to generate a filtered genome file that includes the cancer cell genome data measured at each of the plurality of times.
